(19) United States
(12) Patent Application Publication
(10) Pub. No.: US 2007/0161969 A1
(43) Pub. Date: Jul. 12, 2007

(54) Lock for a guide wire or an intravascular catheter

(76) Inventor: Erik Andersen, Roskiide (DK)

Correspondence Address: BIRCH STEWART KOLASCH & BIRCH, PO BOX 747, FALLS CHURCH, VA, 22040-0747, US

(21) Appl. No.: 10/587,507

(22) PCT Filed: Jan. 28, 2005
(86) PCT No.: PCT/DK05/00071

§ 371 Date: Nov. 13, 2006

Related U.S. Application Data
(60) Provisional application No. 60/539,572, filed on Jan. 29, 2004.

(30) Foreign Application Priority Data
Jan. 29, 2004 (DK) PA 2004 00125

(51) Int. Cl. A61M 39/10; A61M 39/00; A61M 25/16; A61M 25/18

(52) U.S. Cl. 604/533

(57) ABSTRACT

A device for securing one or more guide wires (112,114) or intravascular catheters in relation to a connector (100) of a haemostatic valve assembly as used for example in angioplasty, comprises a frame with a body portion (120) and holders (130;132) for securing the connector in relation to the body portion. A locking mechanism secures the one or more guide wires or catheters in relation to the frame when the guide wires or catheters extend through the connector, the guide wires or catheters being intended to guide balloon catheters and/or stents to an artery treatment site. In one embodiment, the locking mechanism comprises two slidable members (142;144) for pressing the guide wires or catheters against an abutment surface, which may be flexible to provide a secure securing of the guide wires or catheters. Alternatively, there may be provided spring-biased or eccentrically, rotationally mounted locking members.

### TECHNICAL FIELD

The present invention generally relates to the field of connectors for haemostatic valve assemblies, as used for example in angioplasty. More particularly, the invention provides a device for securing a guide wire or a catheter to be introduced into the vascular system of a living being through the connector. Typically, a guide wire is introduced with a catheter and/or a vascular stent. The connector may incorporate a haemostatic valve for safe haemostasis.

### BACKGROUND OF THE INVENTION

Access to the vascular system of a living being, such as a cardiac patient, is required during endovascular procedures such as in angioplasty, e.g., for the introduction of balloon catheters or stent systems. Usually, access is provided via a connector which, e.g., provides a connection to a guiding catheter, the connector integrating a haemostatic valve to enable an elongate device to be introduced into the body of the living being while providing safe haemostasis. A side arm may be provided as a part of such a connector in order to provide a connection to a manifold used for pressure monitoring, contrast media injection and/or saline flushing. Connectors with side arms are normally referred to as ‘Y-connectors’. The haemostatic valve ensures that blood does not flow out of the connector while enabling a catheter, stent system or arterirectomy device to be passed through the connector. At the distal end of the connector there may be provided a rotatable luer for securing the connector to a corresponding member at the proximal end of a guide catheter.

U.S. Pat. No. 5,195,980 (David G. Catlin), discloses a haemostatic valve comprised in a Y-connector. The haemostatic valve is incorporated in a proximal end of a main section of the connector, which comprises a rotatable luer at its distal end. A side arm joins the main section between the distal end and the haemostatic valve.

The art of coronary angioplasty is generally described in: Coronary Angioplasty by Bernhard Meier, published by Grune & Stratton, Inc., Harcourt Brace Jovanovich, Publishers, 1987.

Usually access to the cardiovascular or neurovascular system of a patient is provided at a site remote from the actual treatment site. For example, access to the heart artery is often made via the patient's inguinal artery which is accessed from the outside in the patient's groin region. A guide wire is used for guiding a stent system and/or balloon catheter into place at the treatment site, the guide wire being introduced into the patient's artery system at the remote access site, e.g. the inguinal artery. Great care has to be taken when handling such a guide wire, as precise positioning and careful handling is required in order not to damage the patient's artery walls and in order to correctly position the stent system, balloon or other device at the treatment site. Even greater care is required in case multiple guide wires, which are introduced through one single connector at the same time, are to be handled. Such introduction of multiple guide wires which is, e.g., required for treatment at arterial bifurcations. Proper identification of each of the guide wires is evidently required. Traditionally, identification and handling of guide wires has been performed manually by physicians, such as doctors or nurses. The physicians' handling of guide wires implies a risk of human errors which may severely harm the patient's condition. For example, the identification of two guide wires may be mixed up, or a physician may loose his or her grip to a guide wire which may then cause the stent or balloon to be displaced in an uncontrolled manner inside the patient's artery.

So far, very little has been done to reduce such risks. The present invention therefore seeks to provide a device which can facilitate physicians' handling of guide wires, so as to reduce the risk of human errors during handling thereof.

### SUMMARY OF THE INVENTION

The invention thus provides a device for securing a guide wire or catheter in relation to a connector of a haemostatic valve assembly, comprising:
a frame with a body portion and holding means for securing the connector in relation to the body portion;
a locking mechanism for securing a guide wire or catheter in relation to the frame when the guide wire or catheter extends through the connector.

During a medical procedure, the connector is normally secured in relation to the patient's body by means of a luer lock. It will thus be appreciated that thanks to the provision of the locking mechanism, a physician need not uninterruptedly hold on to a guide wire or catheter during the medical procedure, as the guide wire or catheter may be locked in relation to the frame. The frame is secured in relation to the connector, which in turn is secured in relation to the patient's body. At any time during the procedure, the physician may lock the guide wire or catheter and concentrate on the handling of other equipment, drugs, etc., while the guide wire or catheter is securely locked.

The frame and the connector may define a single, integrated part, or they may define separate parts which are mutually secured. In one embodiment, the frame may support the connector at two different locations along the connector's length in order to provide a firm fixation of the connector to the frame. For example, the frame may form two eyes through which the connector extends. One of the eyes may have the form of a U, into which the connector may be squeezed or snap-fitted, while another one of the eyes may have the form of an O, with a distal portion of the connector having been fitted onto a proximal portion thereof, after introduction of the proximal connector section into the O-shaped eye. The frame may conveniently define a plate-like member, at a distal section of which there is provided the holding means, and at a proximal section of which there is provided the locking mechanism. To facilitate handling of a proximal end of the connector, at which there is usually provided a haemostatic valve assembly, which is to be opened by hand, and through which the guide wires, stent systems etc. are to be introduced, the locking mechanism is preferably arranged at a distance of e.g. 2-8 cm from a proximal end surface of the connector.

The locking mechanism may be adapted to secure at least two guide wires and/or catheters in relation to the frame, preferably so that either one of the two guide wires and/or catheters may be individually locked and released. This may, for example, be achieved by separate locking mechanisms for each of the guide wires and/or catheters. In this case, each of such separate locking mechanisms should be clearly identifiable and distinguishable from the other locking mechanism(s) by a physician handling the guide wires and/or catheters. Such identification may, for example, be achieved by optical identification means, e.g. by different surface colours or textures of the locking mechanisms, and/or by tactile means, e.g. by different surface structures.

In preferred embodiments, the locking mechanism comprises a first locking member which is slidingly arranged in a first slot provided in the frame, and a wall portion protruding from the frame. The slot and the wall preferably coextend with the guide wires and/or catheters, i.e. with the longitudinal direction of the connector and the frame. The slot extends at an oblique angle with respect to the wall portion, and the first locking member is longitudinally movable in the first slot between a first position and a second position. In the first position of the first locking member, the transversal clearance between the wall portion and the first locking member is at most equal to the thickness of the guide wire or catheter, so that the guide wire or catheter is locked between the first locking member and the wall portion. In the second position of the first locking member, the transversal clearance between the wall portion and the first locking members is, however, so large as to allow the guide wire and/or catheter to move in relation to the frame. It may thereby achieved that the guide wire or catheter is securely clamped between the guide wire or catheter and the wall. To allow the device of the invention to function with guide wires and/or catheters of various thicknesses, the slot may extend such with respect to the wall that the clearance between the wall and the first locking member, for example, varies from 0 to 3 mm depending on whether the locking member is in its extreme distal or in its extreme proximal position. The slot may extend such that the smallest clearance between the locking member and the wall exists when the locking member is in a distal position in the slot, i.e. near to the connector, and such the largest clearance between the locking member and the wall exists when the locking member is in a proximal position in the slot, i.e. remote from the connector.

To allow handling and locking of two guide wires or catheters, the locking mechanism may comprise a second locking member which is slidingly arranged in a second slot provided in the frame, the second slot extending at an oblique angle with respect to the wall portion and being arranged at an opposite side of the wall portion with respect to the first slot. Like the first locking member, the second locking member is longitudinally movable in the second slot between first and second positions for locking and releasing a second guide wire or catheter, as explained in detail above in respect of the first locking member and the first slot. Further, similar arrangements may be provided for locking further guide wires or catheters.

In alternative embodiments, the locking mechanism may comprise first, second and/or further locking members biased by compression springs or helical springs for fixing the guide wire or catheter in relation to the frame. Alternative embodiments comprise eccentrically, rotationally mounted locking members.

In embodiments comprising a wall portion against which the operator may press a rotational or sliding locking member, a face of the wall portion facing the guide wire or catheter may be flexible or deformable, so as to more firmly secure the guide wire or catheter when the first and/or second locking member is pressed against the wall portion. In other words, at least a surface portion of the wall portion should be able to deflect or deform slightly when one of the locking members is pressed against it by the operator. For example, the wall portion may be at least partially made from a silicone or rubber material, or at least a portion of the wall portion may be coated with such a material. Likewise, a face of the first locking and/or second member facing the guide wire or catheter may be flexible or deformable and partially made from or coated with a rubber or silicone material.

In the various embodiments of the present invention, the locking members and/or the protruding wall portion may have a surface texture or be coated with a friction-increasing material to increase friction between the locking members and the guide wire and/or catheter.

The present invention also provides, in an independent aspect, an assembly comprising a connector with a haemostatic valve assembly, and a device as claimed and disclosed herein.

### DESCRIPTION OF THE DRAWINGS

Preferred embodiments of the invention will now be further described with reference to the accompanying drawings.

FIG. 1 shows an assembly of a connector 100 and a preferred embodiment of a device 120 for securing two guide wires and/or catheters 112 and 114 in relation to the connector. The connector 100 comprises a proximal section 102 and a distal section 104, the distal section constituting a luer lock know per se. A side arm 106 extends transversely to the longitudinal axis of the connector. A valve 108 defines a proximal end portion of the connector. The securing device 120 comprises a longitudinally extending frame defining a distal section 124, an inclined intermediate section 126, and a proximal section 128 which is at a level different from the level of the distal section to align locking mechanism 134 at the proximal section with the guide wires and/or catheters 112, 114. O- and U-shaped eye portions 130 and 132, respectively, extend in an upright direction from the distal frame section 124 for providing a support for the connector 100. During assembling of the connector and the securing device, the proximal section 102 of the connector is first arranged in the eye portions 130 and 132. Once the proximal section 102 is in place in the eye portions, the distal section 104 is connected and fixed to the proximal section 102, e.g. by snap-fitting or by gluing.

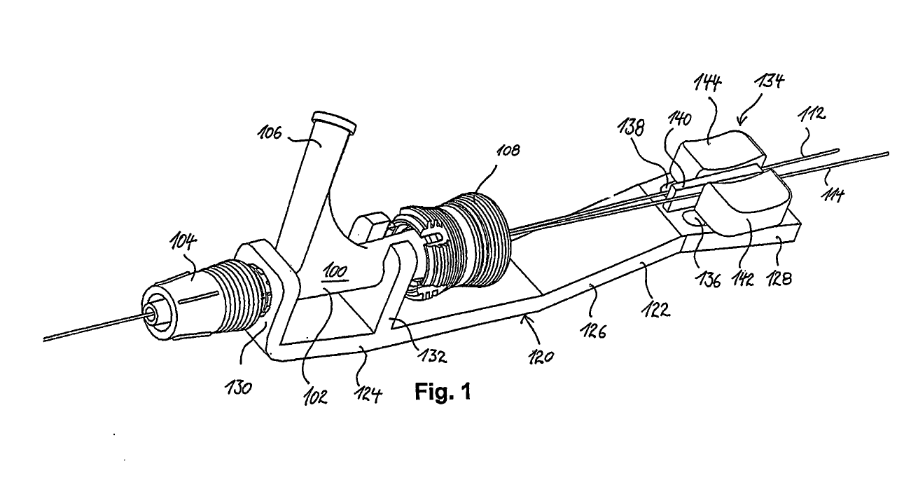

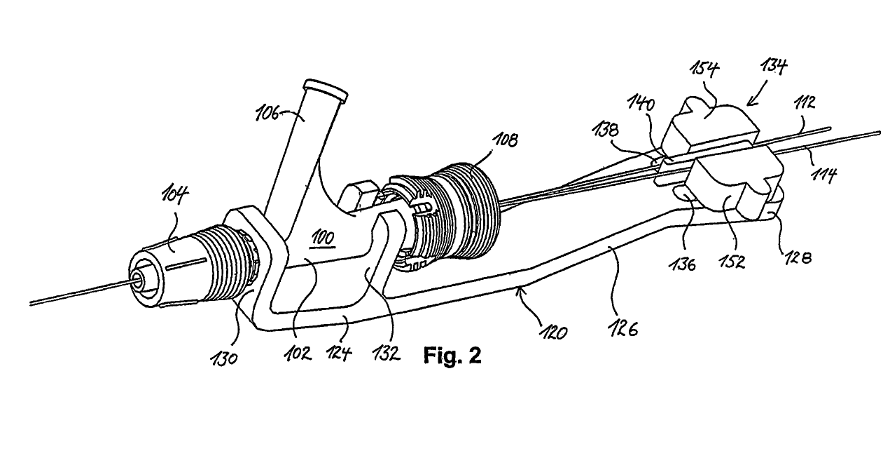

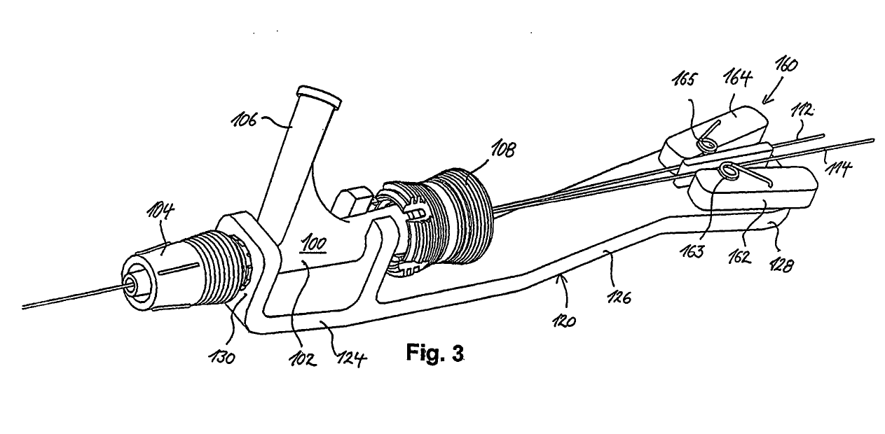

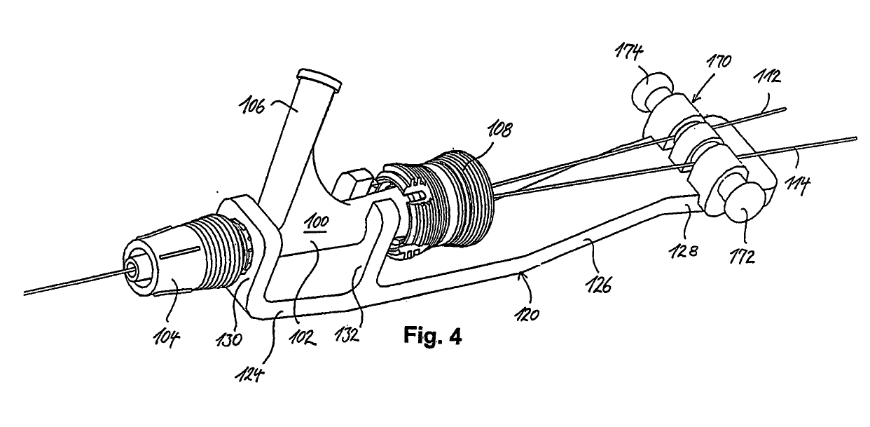

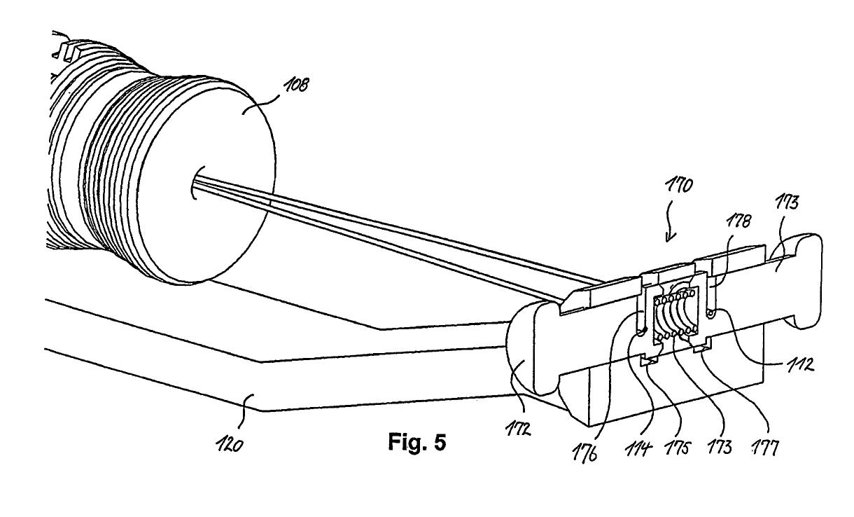

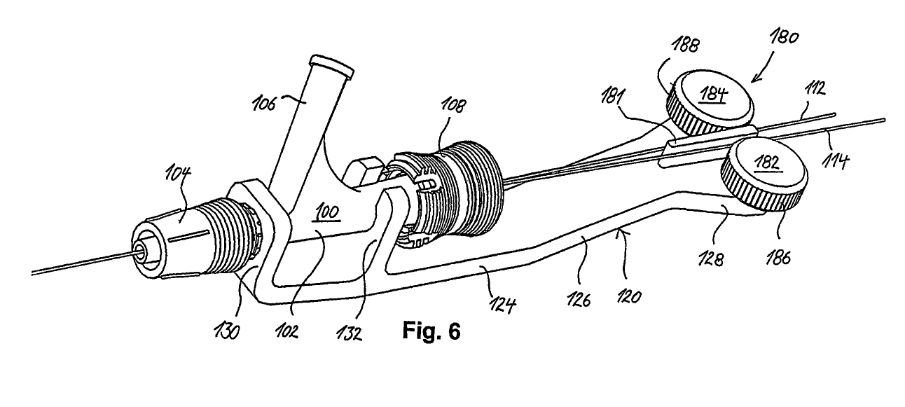

The locking mechanism 134 comprises two slots 136 and 138 extending on either side of a wall portion 140. Each of the slots 136 and 138 define an oblique angle with respect to that wall surface which faces the respective slot. In other words, the slots are non-parallel to their facing wall surfaces. Two locking members 142 and 144 are slidingly arranged in the slots 136 and 138. The guide wires and/or catheters 112 and 114 extend between respective side surfaces of the locking members 142, 144 and left and right side surfaces of the wall. The clearance between the locking members 142, 144 and the side surfaces of the wall is such that the guide wires and/or catheters are firmly secured between the locking members when they are in a distal position in the slots (i.e. to the left in FIG. 1), and such that the guide wires and/or catheters are released and can move in the longitudinal direction when the locking members 142, 144 are in a proximal position (i.e. to the right in FIG. 1). The slots 136 and 138 may be parallel to the longitudinal axis of the connector 100, in which case the side surfaces of the wall portion 140 are non-parallel to the slots 136 and 138. Alternatively, the slots may be non-parallel to the longitudinal axis of the connector and the slots parallel or non-parallel thereto at an angle different from the angle between the wall side surfaces and the slots. The upper surfaces of the locking members 142,144 may be slightly concave to facilitate handling thereof by a physician.

FIG. 2 shows an embodiment similar to the embodiment of FIG. 1 with modified locking members 152 and 154 having protruding portions for improved gripping properties. In FIG. 3, locking mechanism 160 comprises two rotatably mounted locking members 162 and 164 which are spring-biased by spiral springs 163 and 165. The locking mechanism 170 of FIGS. 4 and 5 comprises two transversely displaceable locking members 172 and 174 which are arranged in slits 175 and 177 (see FIG. 5). The locking members 172,174 are spring-biased by compression spring 173, so that the guide wires and/or catheters 112,114 can be firmly secured in grooves 176,178.

In the embodiment of FIG. 6, locking mechanism 180 comprises two rotatably and eccentrically mounted locking members 182 and 184 with rough side surfaces for improved friction properties between the locking members on the one hand and the guide wires (and/or catheters) and physicians' fingers on the other hand. A wall portion 181 extends upwardly from the proximal section 128 of the securing device 120. Due to the eccentric mounting of the locking members 182 and 184, the guide wires and/or catheters 112,114 may move freely between the side surfaces of wall portion 181 and the side surfaces 186,188 of locking members 182,184 when the locking members are in a position in which the side surfaces 186,188 are not close to the wall portion 181, i.e. when the clearance between the side surfaces 186,188 and the wall portion 181 is large enough to allow displacement of the guide wires and/or catheters. When the locking members 182,184, however, are rotated to a position, in which the clearance between the side surfaces 186,188 and the wall portion 181 corresponds to the thickness of the guide wires and/or catheters, and the locking members 182,184 are forced in the direction which further reduces the clearance, the guide wires and/or catheters may be firmly secured.

1. A device for securing a guide wire or an intravascular catheter in relation to a connector of a haemostatic valve assembly, comprising:
a frame with a body portion and holding means for securing the connector in relation to the body portion;
a locking mechanism for securing a guide wire or catheter in relation to the frame when the guide wire or catheters extends through the connector.

2. A device according to claim 1, wherein the locking mechanism is arranged to secure at least two guide wires and/or catheters in relation to the frame.

3. A device according to claim 2, wherein the locking mechanism is arranged to individually lock and release either one the two guide wires and/or catheters.

4. A device according to claim 1, wherein the locking mechanism comprises:
a first locking member which is slidingly arranged in a first slot provided in said frame, and
a wall portion protruding from the frame, the slot extending at an oblique angle with respect to the wall portion, the first locking member being longitudinally movable in said first slot between a first position and a second position, whereby, in the first position of the first locking member, the transversal clearance between the wall portion and the first locking member is at most equal to the thickness of the guide wire or catheter, so that the guide wire or catheter is locked between the first locking member and the wall portion, and whereby, in the second position of the first locking member, the transversal clearance between the wall portion and the first locking members is so large as to allow the guide wire or catheter to move in relation to the frame.

5. A device according to claim 4, wherein the locking mechanism comprises a second locking member which is slidingly arranged in a second slot provided in said frame, the second slot extending at an oblique angle with respect to said wall portion and being arranged at an opposite side of the wall portion with respect to the first slot, the second locking member being longitudinally movable in the second slot between first and second positions for locking and releasing a second guide wire or catheter.

6. A device according to claim 5, wherein at least one of the first and second locking members comprises identification means for distinguishing each of them from the other.

7. A device according to claim 6, wherein the identification means includes different surface colours of the two locking members.

8. A device according to claim 6, wherein the identification means includes tactile means.

9. A device according to claim 4, wherein at least one of:—a face of said wall portion facing the guide wire or catheter; and—a face of said first locking member facing the guide wire or catheter is flexible or deformable.

10. A device according to any of claim 1, wherein the locking mechanism comprises a first and optionally a second spring-biased locking member for fixing the guide wire or catheter in relation to the frame.

11. A device according to claim 1, wherein the locking mechanism comprises a first and optionally a second eccentrically mounted, rotational locking member.

12. A device according to claim 10, wherein at least one of the first and second locking members comprises identification means for distinguishing each of them from the other.

13. An assembly comprising a connector with a haemostatic valve assembly, and a device according to claim 1.

14. Use of a device according to claim 1 for securing a catheter in relation to a connector of a haemostatic valve assembly.
